(19) United States
(12) Patent Application Publication
(10) Pub. No.: US 2023/0293089 A1
(43) Pub. Date: Sep. 21, 2023

(54) BRAIN FUNCTION DETERMINATION APPARATUS, BRAIN FUNCTION DETERMINATION METHOD, AND COMPUTER-READABLE MEDIUM

(71) Applicant: Naohiro OKUMURA, Kanagawa (JP)

(72) Inventor: Naohiro OKUMURA, Kanagawa (JP)

(73) Assignee: Ricoh Company, Ltd., Tokyo (JP)

(21) Appl. No.: 18/176,741

(22) Filed: Mar. 1, 2023

(30) Foreign Application Priority Data
Mar. 18, 2022 (JP) 2022-044158

(51) Int. Cl. A61B 5/00; A61B 5/369; A61B 5/245

(52) U.S. Cl. CPC A61B 5/4076; A61B 5/369; A61B 5/742; A61B 5/245; A61B 5/7264

(57) ABSTRACT

An aspect of the present invention, a brain function determination apparatus includes a first acquisition unit, a first conversion unit, and an identification unit. The first acquisition unit is configured to acquire brain function data including a temporal change, indicating a brain function state measured by a measurement apparatus. The first conversion unit is configured to convert the brain function data acquired by the first acquisition unit, to first converted data including information on at least a time and a space as dimensions. The identification unit is configured to perform an identification process of determining a brain disease and identifying a brain disease region, using the first converted data as an input of a deep learning model constructed by predetermined deep learning.

### CROSS-REFERENCE TO RELATED APPLICATIONS

The present application claims priority under 35 U.S.C. § 119 to Japanese Patent Application No. 2022-044158, filed on Mar. 18, 2022. The contents of which are incorporated herein by reference in their entirety.

### BACKGROUND OF THE INVENTION

### 1. Field of the Invention

The present invention relates to a brain function determination apparatus, a brain function determination method, and a computer-readable medium.

### 2. Description of the Related Art

Due to the influence of declining birthrate and aging population, improvement in life expectancy, and the like, in recent years, the elderly aged 65 and over accounts for about 30% of the total population in Japan. In the accelerated super-aging society, there is an urgent need to increase healthy life expectancy of the people, and dementia is one of issues for which countermeasures need to be taken. As for dementia, it is possible to improve symptoms and slow down the disease to some extent by rehabilitation or medication treatment. However, if once symptoms progress, it is difficult to recover an original state; therefore, as for various kinds of brain diseases including dementia, it is important to detect a disease at an early stage at which no subjective symptom is observed, read a sign at a very early stage, and take preventive measures against the disease.

As a technique for detecting the brain disease at an early stage as described above, there is a known technique for detecting a brain disease of a subject at an early stage by extracting, from brain waves, a feature that is originated from the brain disease, obtaining data by adding, as a label, disease information indicating content of a disease to the extracted feature, and classifying the data by machine learning. In this manner, electro-encephalography data and magneto-encephalography data are information that are close to brain neural activity as compared to brain metabolic rate data or the like, and are widely used in a technique for detecting a brain disease at an early stage.

As a brain disease diagnosis support system capable of determining a brain disease as described above, a certain system is disclosed that obtains electro-encephalography feature data by extracting, from a brain wave, a feature amount of the brain wave, acquires a plurality of pieces of learning data in each of which disease information indicating a brain disease corresponding to the electroencephalography feature data is added to the electroencephalography feature data, classifies the pieces of acquired learning data into a plurality of clusters, generates a classifier that classifies the learning data for each piece of the disease information based on the disease information added to the learning data in each of the classified clusters, acquires electro-encephalography feature data of a subject, identifies a cluster into which the electroencephalography feature data of the subject is classified, and determines a brain disease corresponding to the electro-encephalography feature data of the subject from among a plurality of brain diseases by the generated classifier (for example, Japanese Unexamined Patent Application Publication No. 2016-106940).

However, the conventional technique for detecting a brain disease at an early stage is based on the assumption that a feature is extracted, and it is difficult to perform derivation through an analysis based on multidimensional data, and therefore, it is difficult to accurately determine a brain disease and identify a brain disease region from data including a temporal change, which is a problem.

### SUMMARY OF THE INVENTION

According to an aspect of the present invention, a brain function determination apparatus includes a first acquisition unit, a first conversion unit, and an identification unit. The first acquisition unit is configured to acquire brain function data including a temporal change, indicating a brain function state measured by a measurement apparatus. The first conversion unit is configured to convert the brain function data acquired by the first acquisition unit, to first converted data including information on at least a time and a space as dimensions. The identification unit is configured to perform an identification process of determining a brain disease and identifying a brain disease region, using the first converted data as an input of a deep learning model constructed by predetermined deep learning.

### BRIEF DESCRIPTION OF THE DRAWINGS

FIG. 1 is a schematic configuration diagram of a brain function determination system according to one embodiment;

FIG. 2 is a diagram illustrating an example of a hardware configuration of an information processing apparatus according to the embodiment;

FIG. 3 is a diagram illustrating an example of a functional block configuration of the information processing apparatus according to the embodiment;

FIGS. 4A and 4B are diagrams for explaining an overview of entire operation of the brain function determination system according to the embodiment;

FIG. 5 is a flowchart illustrating an example of the flow of the entire operation of the brain function determination system according to the embodiment; and

FIG. 6 is a diagram illustrating an example of a screen in which a brain disease region that is identified through an identification process performed by the information processing apparatus according to the embodiment is visualized.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

Embodiments of a brain function determination apparatus, a brain function determination method and a computer-readable medium according to the present invention will be described in detail below with reference to the drawings. The present invention is not limited by the embodiments below, and components in the embodiments below include one that can easily be thought of by a person skilled in the art, one that is practically identical, one that is what is called an equivalent, and the like. Furthermore, various omission, replacement, modifications, and combinations of the components may be made without departing from the gist of the embodiments described below.

An embodiment has an object to provide a brain function determination apparatus, a brain function determination method, and a computer-readable medium capable of accurately determining a brain disease and identifying a brain disease region from data including a temporal change.

Overview of Brain Function Determination System

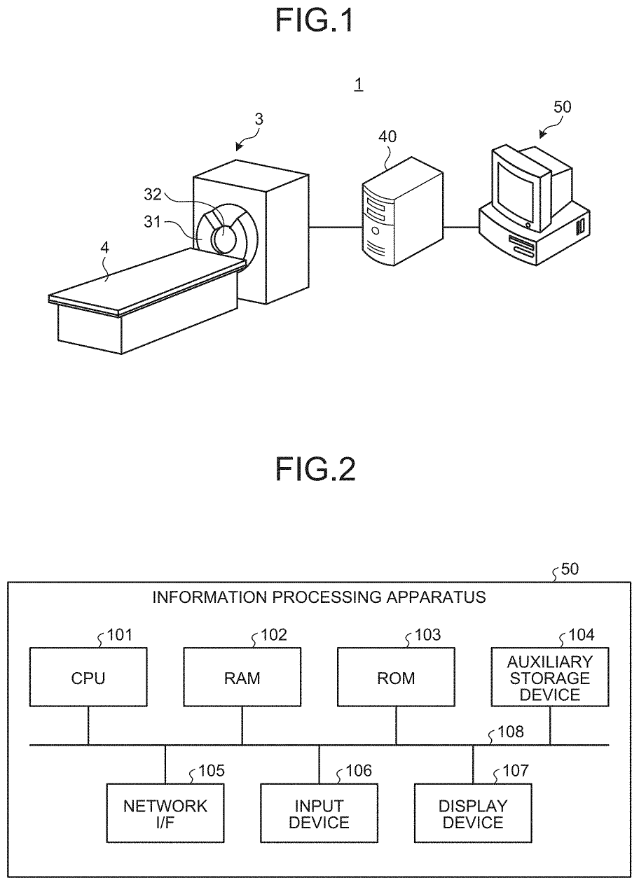

FIG. 1 is a schematic configuration diagram of a brain function determination system according to one embodiment. An overview of a brain function determination system 1 according to the present embodiment will be described below with reference to FIG. 1.

The brain function determination system 1 is a system that measure and acquires brain function imaging data (one example of brain function data) that is a plurality of kinds of biological signals (for example, magneto-encephalography (MEG) data, electro-encephalography (EEG) data, and the like) of a subject, determines a brain disease, identifies a brain disease region, and visualizes a portion corresponding to the brain disease on data or a brain image. The brain function imaging data is data that includes a temporal change and that is obtained by measuring physiologically active (function) state of each of portions in the brain by various kinds of methods. Meanwhile, the biological signal as the brain function imaging data that is a measurement target is not limited to data that includes the magneto-encephalography data and the electro-encephalography data.

As illustrated in FIG. 1, the brain function determination system 1 includes a measurement apparatus 3 that measures one or more kinds of brain function imaging data of a subject, a server 40 that accumulates the one or more kinds of brain function imaging data measured by the measurement apparatus 3, and an information processing apparatus 50 (brain function determination apparatus) that analyzes the one or more kinds of brain function imaging data recorded in the server 40. Meanwhile, in FIG. 1, the server 40 and the information processing apparatus 50 are illustrated as separate apparatuses; however, for example, at least a part of functions of the server 40 may be incorporated in the information processing apparatus 50. Furthermore, in FIG. 1, the information processing apparatus 50 is illustrated as a single information processing apparatus, but embodiments are not limited to this example, and an information processing system (one example of the brain function determination system) that includes a plurality of information processing apparatuses may be applicable.

In the example illustrated in FIG. 1, a subject (to-be-measured person) lies down on a measurement table 4 with face up while electrodes (or sensors) for electroencephalography are mounted on his/her head, and a head portion is inserted in a hollow 32 of a dewar 31 of the measurement apparatus 3. The dewar 31 is a holding container in an extremely low temperature environment using liquid helium, and a large number of magnetic sensors for magnetoencephalography are arranged inside the hollow 32 of the dewar 31. The measurement apparatus 3 collects electro-encephalography data from the electrodes and magneto-encephalography data from the magnetic sensors, and outputs brain function imaging data that includes the collected electro-encephalography data and the collected magneto-encephalography data to the server 40. The brain function imaging data that is output to the server 40 is read, displayed, and analyzed by the information processing apparatus 50. In general, the dewar 31 in which the magnetic sensors are incorporated and the measurement table 4 are arranged in a magnetic shielding room, but illustration of the magnetic shielding room is omitted in FIG. 1 for the sake of convenience.

The information processing apparatus 50 is an apparatus that analyzes the magneto-encephalography data obtained from the plurality of magnetic sensors and the electro-encephalography data obtained from the plurality of electrodes. The electro-encephalography data is a signal that represents electrical activity of a nerve cell (ion charge flow that occurs in dendrites of a neuron at the time of synaptic transmission) as a voltage value between the electrodes. The magneto-encephalography data is a signal that represents minute magnetic field variation that occurs due to electrical activity of a brain. The brain's magnetic field is detected by a high-sensitive superconducting quantum interference device (SQUID) sensor. The electro-encephalography data and the magneto-encephalography data are one example of a “biological signal” and “brain function imaging data”.

Hardware Configuration of Information Processing Apparatus

FIG. 2 is a diagram illustrating an example of a hardware configuration of the information processing apparatus according to the embodiment. The hardware configuration of the information processing apparatus 50 according to the present embodiment will be described below with reference to FIG. 2.

As illustrated in FIG. 2, the information processing apparatus 50 includes a central processing unit (CPU) 101, a random access memory (RAM) 102, a read only memory (ROM) 103, an auxiliary storage device 104, a network interface (I/F) 105, an input device 106, and a display device 107, all of which are connected to one another via a bus 108.

The CPU 101 is an arithmetic device that controls entire operation of the information processing apparatus 50 and performs various kinds of information processing. The CPU 101 executes a program that is stored in the ROM 103 or the auxiliary storage device 104 and controls a learning process and an identification process using deep learning (to be described later) and display operation, such as visualization of an identification result.

The RAM 102 is a volatile storage device that is used as a work area of the CPU 101 and that stores therein main control parameters and information. The ROM 103 is a non-volatile storage device that stores therein a basic input-output program or the like. For example, it may be possible to store the program as described above in the ROM 103.

The auxiliary storage device 104 is a non-volatile storage device, such as a hard disk drive (HDD) or a solid state drive (SSD). The auxiliary storage device 104 stores therein, for example, a program for controlling the operation of the information processing apparatus 50, various kinds of data and files that are needed for the operation of the information processing apparatus 50, and the like.

The network I/F 105 is a communication interface for performing communication with an apparatus, such as the server 40, on a network. The network I/F 105 is implemented by, for example, a network interface card (NIC) or the like that is compliant with transmission control protocol/Internet protocol (TCP/IP).

The input device 106 is an input function of a touch panel, a user interface, such as a keyboard, a mouse, or an operation button, or the like. The display device 107 is a display device that displays various kinds of information. The display device 107 is implemented by, for example, a display function of a touch panel, a liquid crystal display (LCD), an organic electro-luminescence (EL), or the like.

Meanwhile, the hardware configuration of the information processing apparatus 50 illustrated in FIG. 2 is one example, and a different device may be added. Further, the information processing apparatus 50 illustrated in FIG. 2 has the hardware configuration based on the assumption that the information processing apparatus 50 is a personal computer (PC) for example, but embodiments are not limited to this example, and a mobile terminal, such as a tablet, may be adopted. In this case, it is sufficient that the network I/F 105 is a communication interface with a wireless communication function.

Functional Block Configuration and Operation of Information Processing Apparatus

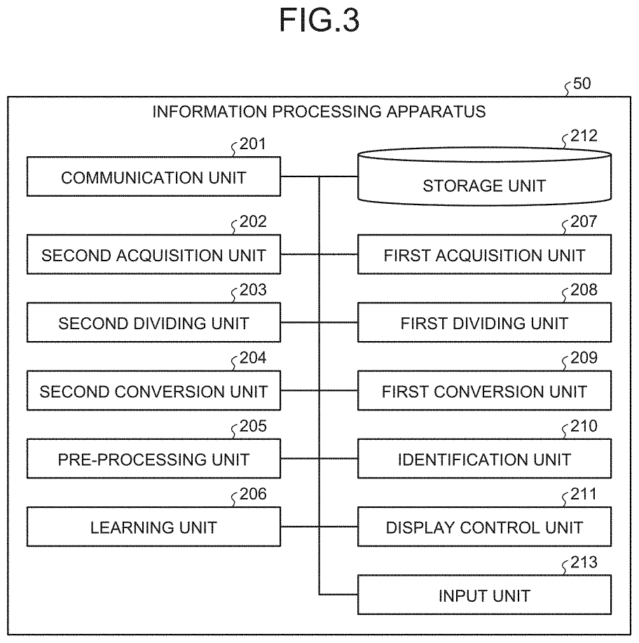

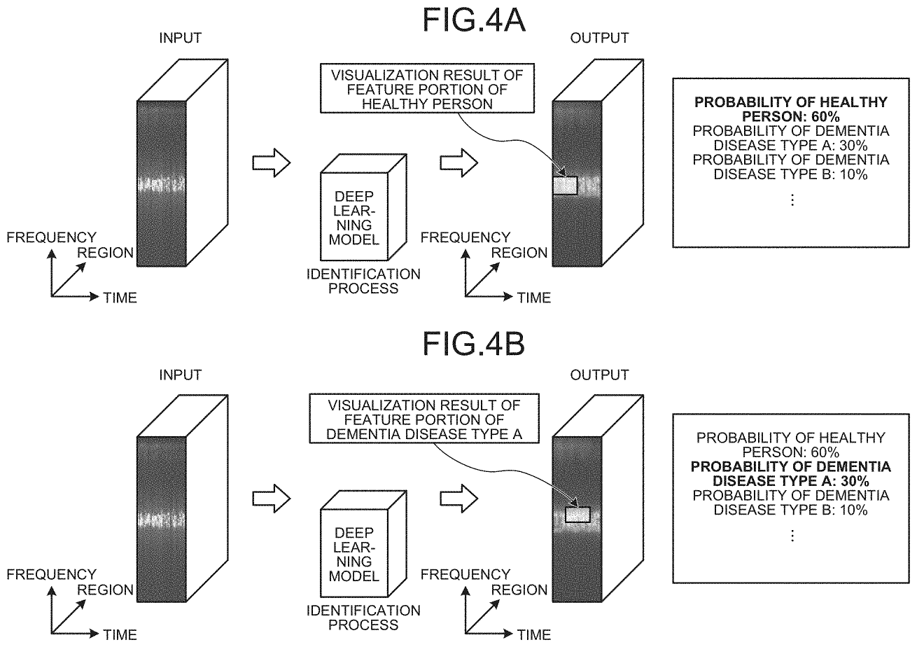

FIG. 3 is a diagram illustrating an example of a functional block configuration of the information processing apparatus according to the embodiment. FIGS. 4A and 4B are diagrams for explaining an overview of entire operation of the brain function determination system according to the embodiment. The functional block configuration and the operation of the information processing apparatus 50 according to the present embodiment will be described below with reference to FIG. 3 and FIGS. 4A and 4B.

As illustrated in FIG. 3, the information processing apparatus 50 includes a communication unit 201, a second acquisition unit 202, a second dividing unit 203, a second conversion unit 204, a pre-processing unit 205 (standardization unit), a learning unit 206, a first acquisition unit 207, a first dividing unit 208, a first conversion unit 209, an identification unit 210, a display control unit 211, a storage unit 212, and an input unit 213.

The communication unit 201 is a functional unit that performs data communication with the measurement apparatus 3, the server 40, or the like. For example, the communication unit 201 receives the brain function imaging data from the server 40 and stores the brain function imaging data in the storage unit 212. Meanwhile, the communication unit 201 may directly receive the brain function imaging data from the measurement apparatus 3. The communication unit 201 is implemented by the network I/F 105 illustrated in FIG. 2.

The second acquisition unit 202 is a functional unit that acquires the brain function imaging data that is received by the communication unit 201. In this case, the brain function imaging data that is acquired by the second acquisition unit 202 has a disease label added, the disease label indicating content of a brain disease or a healthy state, and is used as learning data (hereinafter, may be referred to as training data) that is used for a learning process of deep learning by the learning unit 206. Meanwhile, the second acquisition unit 202 need not always acquire the brain function imaging data from the communication unit 201, but may acquire the brain function imaging data that is stored in the storage unit 212.

The second dividing unit 203 is a functional unit that performs an epoching process of dividing the brain function imaging data that is acquired by the second acquisition unit 202 by an arbitrary time interval (time window).

The second conversion unit 204 is a functional unit that converts the brain function imaging data that has been divided by the second dividing unit 203 into data (hereinafter, may be referred to as converted data) (second converted data) that includes information on at least a time and a space as dimensions. For example, the second conversion unit 204 is able to obtain converted data that includes information on signal intensity (power) based on an amplitude, a frequency, a space, and a time as dimensions by performing frequency conversion based on the Fourier transform or the like for each channel and each division for which the brain function imaging data is measured. By performing the conversion by the second conversion unit 204 as described above, it is possible to obtain the converted data without losing a feature of the brain function imaging data. Meanwhile, it may be possible to use the brain function imaging data as it is in a learning process performed by the learning unit 206 on the subsequent stage, and, in this case, the second conversion unit 204 performs identity transform as the conversion. Furthermore, examples of the conversion process performed by the second conversion unit 204 include extraction and enhancement of a sensor, down-sampling, application of a frequency filter, elimination of artifacts, a defective channel process, extraction of a time window, and standardization of magnetic field data.

The pre-processing unit 205 is a functional unit that performs a predetermined standardization process on the converted data that is obtained by the second conversion unit 204 because the brain function imaging data is multidimensional and a data scale varies. Examples of the standardization process include a process of aligning ranges of the converted data for which the ranges are different, and, with this process, it becomes possible to stabilize the learning process performed by the learning unit 206 on the subsequent stage.

Meanwhile, the learning data that is divided by the second dividing unit 203, the converted data that is converted by the second conversion unit 204, and the converted data that is subjected to the standardization process by the pre-processing unit 205 may also be referred to as the training data, in addition to the brain function imaging data that is acquired by the second acquisition unit 202, because these pieces of the data are used for the learning process performed by the learning unit 206.

The learning unit 206 is a functional unit that performs the learning process, using the converted data, which is subjected to the standardization process by the pre-processing unit 205 and to which the disease label is added, as an input through deep learning with a time series analysis function. For example, the learning unit 206 performs a learning process by internally constructing a neural network based on an algorithm, such as a convolutional neural network (CNN), to extract a feature on spatial information, and constructing a neural network based on an algorithm, such as a recurrent neural network (RNN) or an attention, to extract a feature on temporal information. With this configuration, it is possible to extract a feature that is peculiar to a brain disease while emphasizing a brain region and a time, so that it is possible to construct a neural network capable of accurately determining the brain disease. Meanwhile, in the feature extraction as described above, it is not needed for a human being to define a type of feature data to be extracted from the learning data in advance as in the machine learning, but, in the deep learning, a type of the feature data to be extracted from the learning data is automatically determined during the learning process. Furthermore, construction of the neural network indicates, in particular, a process of adjusting and determining a weight or the like that is strength of synaptic connections in the neural network. The neural network (hereinafter, may be referred to as a deep learning model) that is constructed through the learning process performed by the learning unit 206 is stored in the storage unit 212. Specifically, data of the determined weight or the like for the neural network is stored in the storage unit 212. In this manner, with use of the deep learning model that is obtained through the learning process performed by the learning unit 206, it becomes possible to determine presence or absence of a brain disease, such as dementia, developmental disorders, or psychosis, determine a brain disease, determine a disease type, and identify a brain disease region.

The first acquisition unit 207 is a functional unit that acquires the brain function imaging data that is received by the communication unit 201. In this case, the brain function imaging data that is acquired by the first acquisition unit 207 is data for which a type of a brain disease is to be identified, to which the disease label is not added, and which is used as data (hereinafter, may be referred to as visualized data) for performing an identification process using the deep learning model and visualizing a result of the identification. Meanwhile, the first acquisition unit 207 need not always acquire the brain function imaging data from the communication unit 201, but may acquire the brain function imaging data that is stored in the storage unit 212.

The first dividing unit 208 is a functional unit that performs an epoching process of dividing the brain function imaging data that is acquired by the first acquisition unit 207 by an arbitrary time interval (time window).

The first conversion unit 209 is a functional unit that converts the brain function imaging data that is divided by the first dividing unit 208 into data (hereinafter, may be referred to as converted data) (first converted data) that includes information on at least a time and a space as dimensions. For example, the first conversion unit 209 is able to obtain converted data that includes information on signal intensity (power) based on an amplitude, a frequency, a space, and a time as dimensions by performing frequency conversion based on the Fourier transform or the like for each channel and each division for which the brain function imaging data is measured. By performing the first conversion unit 209 as described above, it is possible to obtain the converted data without losing a feature of the brain function imaging data. Meanwhile, it may be possible to use the brain function imaging data as it is in a learning process performed by the identification unit 210 on the subsequent stage, and, in this case, the first conversion unit 209 performs identity transform as the conversion. Furthermore, examples of the conversion process performed by the first conversion unit 209 include extraction and enhancement of a sensor, down-sampling, application of a frequency filter, elimination of artifacts, a defective channel process, extraction of a time window, and standardization of magnetic field data.

The converted data obtained by the first conversion unit 209 is, as illustrated in FIGS. 4A and 4B, data that is to be input to the deep learning model that is constructed through the learning process performed by the learning unit 206. In the example illustrated in FIGS. 4A and 4B, signal intensity is illustrated by a heat map in a three-dimensional region in which a horizontal axis represents a time, a vertical axis represents a frequency, and a depth represents a space (region). Here, the space as the depth indicates a brain region, such as a frontal lobe, a temporal lobe, or an occipital lobe of the brain, which is determined in advance, and each brain region is associated with the depth axis for the sake of convenience.

Meanwhile, the data that is divided by the first dividing unit 208, the converted data that is converted by the first conversion unit 209, and the converted data that is subjected to the standardization process by the pre-processing unit 205 may be referred to as, in addition to the brain function imaging data that is acquired by the first acquisition unit 207, visualized data because these pieces of data are data that are used for the identification process using the deep learning model by the identification unit 210 and that are to be visualized as the identification result.

The identification unit 210 is a functional unit that reads the deep learning model that is constructed through the learning process performed by the learning unit 206 from the storage unit 212, and performs the identification process, using the converted data obtained by the first conversion unit 209 as an input for the deep learning model. Here, in particular, the identification process indicates a process of determining presence or absence of a brain disease, determining a brain disease, determining a disease type, and identifying a brain disease region, using the deep learning model. Meanwhile, the identification unit 210 may input, as a result of the identification process, the converted data to the deep learning model, and obtain probabilities of each disease type of each brain disease, such as dementia, and a healthy state, or may be able to calculate and obtain the probabilities based on an output of the deep learning model. Furthermore, the converted data obtained by the first conversion unit 209 may be subjected to the standardization process in the same manner as performed by the pre-processing unit 205, and may thereafter be input to the deep learning model.

The display control unit 211 is a functional unit that causes the display device 107 to display, as the identification result obtained by the identification unit 210, a result of presence or absence of a brain disease, a determination result of the brain disease, a determination result of a disease type, an identified brain disease region, and the like. For example, as illustrated in FIGS. 4A and 4B, the display control unit 211 may visualize a data portion that is a basis for the determination on the brain disease and the disease type by enclosing the data portion by a rectangle or the like on the heat map that is arranged in the three-dimensional region in which the horizontal axis represents a time, the vertical axis represents a frequency, and the depth represents a space (region). In the example illustrated in FIG. 4A, if the probability of the healthy state is calculated as 60% as the identification result, the display control unit 211 visualizes a data portion indicating a feature portion as the healthy state by enclosing the data portion by a rectangle or the like on the heat map of the signal intensity of a specific brain region (depth). Furthermore, in the example illustrated in FIG. 4B, if the probability of a disease type A, which indicates dementia, is calculated as 30% as the identification result, the display control unit 211 visualizes a data portion indicating a feature portion as the disease type A by enclosing the data portion by a rectangle or the like on the heat map of the signal intensity of a specific brain region (depth). In other words, the display control unit 211 is able to visualize the identification result obtained by the deep learning model for each brain disease (or healthy state). Moreover, as illustrated in FIG. 4A and FIG. 4B, the display control unit 211 may display, as the identification results, the probabilities of the healthy state and each disease type of each brain disease. By visualization of the identification result by the display control unit 211 as described above, it is possible to indicate, on the visualized data, a data portion of a time, a frequency, a brain region, and signal intensity that are identified as a basis for the determination on the brain disease or the healthy state. In other words, it is possible to visualize the identification result with respect to the same-dimensional data as the visualized data (converted data) that is input to the deep learning model, so that it is possible to identify a brain disease region that is less likely to be affected by a temporal change.

The storage unit 212 is a functional unit that stores therein the brain function imaging data that is received by the communication unit 201, the deep learning model that is constructed through the learning process performed by the learning unit 206, and the like. The storage unit 212 is implemented by the RAM 102 or the auxiliary storage device 104 illustrated in FIG. 2.

The second acquisition unit 202, the second dividing unit 203, the second conversion unit 204, the pre-processing unit 205, the learning unit 206, the first acquisition unit 207, the first dividing unit 208, the first conversion unit 209, the identification unit 210, and the display control unit 211 as described above are implemented by causing the CPU 101 to load a program that is stored in the ROM 103 or the like onto the RAM 102 and execute the loaded program. Meanwhile, a part or all of the second acquisition unit 202, the second dividing unit 203, the second conversion unit 204, the pre-processing unit 205, the learning unit 206, the first acquisition unit 207, the first dividing unit 208, the first conversion unit 209, the identification unit 210, and the display control unit 211 may be implemented by a hardware circuit, such as an application specific integrated circuit (ASIC) or a field-programmable gate array (FPGA), instead of a program that is software.

Meanwhile, each of the functional units illustrated in FIG. 3 is a functionally conceptual, and need not always be configured in the same manner. For example, a plurality of functional units that are illustrated as independent functional units in FIG. 3 may be configured as a signal functional unit. In contrast, a function included in a single functional unit in FIG. 3 may be divided into a plurality of functions, and may be configured as a plurality of functional units.

Furthermore, in the information processing apparatus 50 illustrated in FIG. 3, it is assumed that the learning process through the deep learning using the brain function imaging data and the identification process using the deep learning model are performed in the same apparatus, but embodiments are not limited to this example. For example, the learning process through the deep learning may be performed by an external apparatus (one example of a second apparatus) that is different from the information processing apparatus 50 (one example of a first apparatus). In this case, it is sufficient that the external apparatus includes at least the same functional units as the second acquisition unit 202, the second dividing unit 203, the second conversion unit 204, the pre-processing unit 205, and the learning unit 206.

Entire Operation of Brain Function Determination System

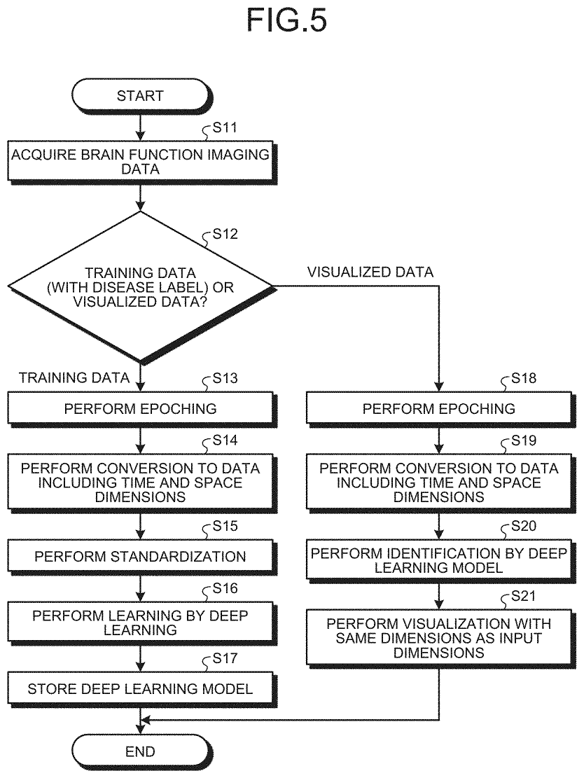

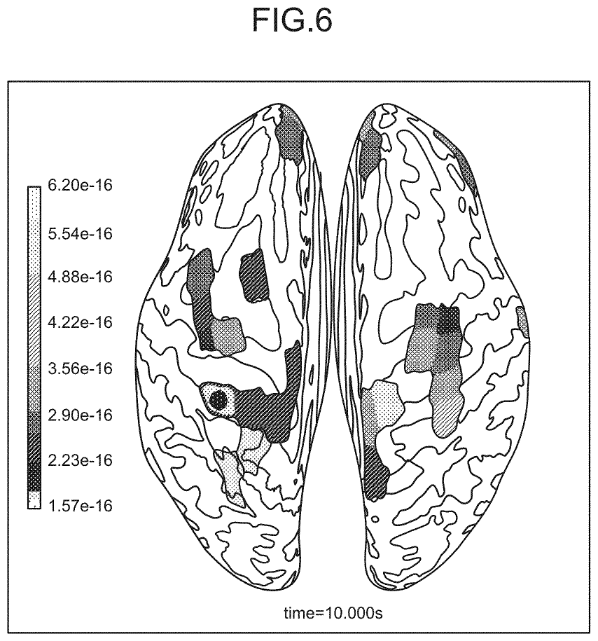

FIG. 5 is a flowchart illustrating an example of the flow of the entire operation of the brain function determination system according to the embodiment. FIG. 6 is a diagram illustrating an example of a screen in which a brain disease region that is identified through the identification process performed by the information processing apparatus according to the embodiment is visualized. The flow of the entire operation of the brain function determination system 1 according to the present embodiment will be described below with reference to FIG. 5 and FIG. 6.

Step S11

The information processing apparatus 50 receives (acquires) the brain function imaging data from the communication unit 201. Meanwhile, the information processing apparatus 50 may read the stored brain function imaging data that is the brain function imaging data received in advance. Then, the process goes to Step S12.

Step S12

If the brain function imaging data that is received (acquired) by the information processing apparatus 50 is the training data to which the disease label is added (Step S12: training data), the second acquisition unit 202 acquires the brain function imaging data, and the process goes to Step S13. In contrast, if the brain function imaging data that is received (acquired) by the information processing apparatus 50 is the visualized data to which the disease label is not added (Step S12: the visualized data), the first acquisition unit 207 acquires the brain function imaging data, and the process goes to Step S18.

Step S13

The second dividing unit 203 of the information processing apparatus 50 performs an epoching process of dividing the brain function imaging data that is acquired by the second acquisition unit 202 by an arbitrary time interval (time window). Then, the process goes to Step S14.

Step S14

The second conversion unit 204 of the information processing apparatus 50 converts the brain function imaging data that is divided by the second dividing unit 203 into data (converted data) that includes information on at least a time and a space as dimensions. Then, the process goes to Step S15.

Step S15

The pre-processing unit 205 of the information processing apparatus 50 performs a predetermined standardization process on the converted data that is obtained by the second conversion unit 204 because the brain function imaging data is multidimensional and a data scale varies. Then, the process goes to Step S16.

Step S16

The learning unit 206 of the information processing apparatus 50 performs a learning process, using the converted data, which is subjected to the standardization process by the pre-processing unit 205 and to which the disease label is added, as an input through deep learning with a time series analysis function. For example, the learning unit 206 performs a learning process by internally constructing a neural network based on an algorithm, such as a CNN, to extract a feature on spatial information, and constructing a neural network based on an algorithm, such as an RNN or an attention, to extract a feature on temporal information. Then, the process goes to Step S17.

Step S17

The deep learning model that is constructed through the learning process performed by the learning unit 206 is stored in the storage unit 212. Specifically, data of the determined weight or the like for the neural network is stored in the storage unit 212. Through the flow as described above, the learning process in the operation of the brain function determination system 1 is terminated.

Step S18

The first dividing unit 208 of the information processing apparatus 50 performs an epoching process of dividing the brain function imaging data that is acquired by the first acquisition unit 207 by an arbitrary time interval (time window). Then, the process goes to Step S19.

Step S19

The first conversion unit 209 of the information processing apparatus 50 converts the brain function imaging data that is divided by the first dividing unit 208 into data (converted data) that includes information on at least a time and a space as dimensions. Then, the process goes to Step S20.

Step S20

The identification unit 210 of the information processing apparatus 50 reads the deep learning model that is constructed through the learning process performed by the learning unit 206 from the storage unit 212, and performs the identification process, using the converted data obtained by the first conversion unit 209 as an input for the deep learning model. Then, the process goes to Step S21.

Step S21

The display control unit 211 of the information processing apparatus 50 causes the display device 107 to display, as the identification result obtained by the identification unit 210, a result of presence or absence of a brain disease, a determination result of the brain disease, a determination result of a disease type, an identified brain disease region, and the like. For example, the display control unit 211 may visualize a data portion that is a basis for the determination on the brain disease and the disease type by enclosing the data portion by a rectangle or the like on a heat map that is arranged in the three-dimensional region in which the horizontal axis represents a time, the vertical axis represents a frequency, and the depth represents a space (region). Further, the display control unit 211 may display, as the identification result, the probability of the healthy state or each disease type of each brain disease. Furthermore, as illustrated in FIG. 6, the display control unit 211 may display, as the identification result, a heat map that represents a time and signal intensity of a frequency selected by a user based on the converted data that is the basis for the brain disease determined by the identification unit 210, to be superimposed on a corresponding brain disease region on a brain image. Moreover, it may be possible to allow the user to select a brain disease (or a healthy state) to be visualized. With this configuration, it is possible to display distributions of a time, a frequency, a brain region, and signal intensity that are identified as the basis for the determination of the brain disease on the brain image.

As described above, in the brain function determination system 1 according to the present embodiment, the first acquisition unit 207 acquires the brain function imaging data that is measured by the measurement apparatus 3, the first conversion unit 209 converts the brain function imaging data that is acquired by the first acquisition unit 207 to converted data that includes information on at least a time and a space as dimensions, the identification unit 210 performs the identification process of determining a brain disease and identifying a brain disease region, using the converted data as an input of a deep learning model that is constructed by predetermined deep learning. With this configuration, it is possible to accurately determine a brain disease and identify a brain disease region from data including a temporal change.

Furthermore, in the brain function determination system 1 according to the present embodiment, the display control unit 211 causes the display device 107 to display an identification result of the identification process performed by the identification unit 210. With this configuration, it is possible to recognize the determination result of the brain disease and the identified brain disease region.

Moreover, in the brain function determination system 1 according to the present embodiment, the display control unit 211 displays, as the identification result obtained by the identification unit 210, a data portion that is a basis for determination on the brain disease or the healthy state such that the data portion on the converted data is identifiable. By the visualization of the identification result by the display control unit 211, it is possible to indicate, on the visualized data, a data portion of a time, a frequency, a brain region, and signal intensity that are identified as the basis for the determination on the brain disease or the healthy state.

Furthermore, in the brain function determination system 1 according to the present embodiment, the display control unit 211 displays the identification result with respect to the same-dimensional data as the converted data that is input to the deep learning model. With this configuration, it is possible to visualize the identification result with respect to the same-dimensional data as the visualized data (converted data) that is input to the deep learning model.

Moreover, in the brain function determination system 1 according to the present embodiment, the display control unit 211 displays, as the identification result, a heat map that represents a specific time and signal intensity of a frequency, to be superimposed on a corresponding brain disease region of a brain image, for each of specific brain diseases. With this configuration, it is possible to display distributions of a time, a frequency, a brain region, and signal intensity that are identified as the basis for the determination of the brain disease on the brain image.

Furthermore, in the brain function determination system 1 according to the present embodiment, the identification unit 210 calculates, as the identification result, probabilities of each disease type of each brain disease and the healthy state based on an output of the deep learning model, and the display control unit 211 displays the probabilities. With this configuration, it is possible to recognize the probabilities of each disease type of each brain disease and the healthy state.

Moreover, in the brain function determination system 1 according to the present embodiment, the second acquisition unit 202 acquires the brain function imaging data that is measured by the measurement apparatus 3 and that has the disease label added the disease label indicating content of a brain disease or a healthy state, the second conversion unit 204 converts the brain function imaging data that is acquired by the second acquisition unit 202 to converted data that includes information on at least a time and a space as dimensions, and the learning unit 206 constructs a deep learning model through a learning process based on the deep learning, using the converted data to which the disease label is added as an input. With this configuration, it is possible to construct a deep learning model that is able to accurately determine a brain disease and identify a brain disease region from data including a temporal change.

Furthermore, in the brain function determination system 1 according to the present embodiment, the pre-processing unit 205 performs a predetermined standardization process on the converted data, and the learning unit 206 constructs the deep learning model, using the converted data that is subjected to the standardization process as an input. With this configuration, it is possible to stabilize learning using deep learning.

Moreover, in the brain function determination system 1 according to the present embodiment, the deep learning model is constructed by deep learning with a time series analysis function. With this configuration, it is possible to process data including various kinds of temporal changes with high accuracy.

Meanwhile, in the embodiment as described above, if at least one of the functional units of the brain function determination system 1 (the information processing apparatus 50) is implemented by execution of a program, the program is provided by being incorporated in a ROM or the like in advance. Further, the program that is executed by the brain function determination system 1 (the information processing apparatus 50) according to the embodiment as described above may be provided by being recorded in a computer readable recording medium, such as a compact disc (CD)-ROM, a flexible disk (FD), a compact disk recordable (CD-R), or a digital versatile disk, in a computer-installable or computer-executable file format. Furthermore, the program that is executed by the brain function determination system 1 (the information processing apparatus 50) of the embodiment as described above may be provided by being stored in a computer that is connected to a network, such as the Internet, and by being downloaded via the network. Moreover, the program that is executed by the brain function determination system 1 (the information processing apparatus 50) of the embodiment as described above may be provided or distributed via a network, such as the Internet. Furthermore, the program that is executed by the brain function determination system 1 (the information processing apparatus 50) of the embodiment as described above has a module structure that includes at least any of the functional units as described above, and as an actual hardware, each of the functional units as described above is loaded and generated on a main storage device by causing the CPU to read the program from the ROM or the like.

According to an embodiment, it is possible to accurately determine a brain disease and identify a brain disease region from data including a temporal change.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A brain function determination apparatus comprising:
a first acquisition unit configured to acquire brain function data including a temporal change, indicating a brain function state measured by a measurement apparatus;
a first conversion unit configured to convert the brain function data acquired by the first acquisition unit, to first converted data including information on at least a time and a space as dimensions; and
an identification unit configured to perform an identification process of determining a brain disease and identifying a brain disease region, using the first converted data as an input of a deep learning model constructed by predetermined deep learning.

2. The brain function determination apparatus according to claim 1, further comprising a display control unit configured to display, on a display device, an identification result of the identification process by the identification unit.

3. The brain function determination apparatus according to claim 2, wherein the display control unit is configured to display the identification result by the identification unit such that a data portion on the first converted data is identifiable, the data portion being a basis for determination on one of a brain disease and a healthy state.

4. The brain function determination apparatus according to claim 2, wherein the display control unit is configured to display the identification result with respect to same-dimensional data as the first converted data being the input to the deep learning model.

5. The brain function determination apparatus according to claim 2, wherein the display control unit is configured to display, as the identification result, a heat map representing a specific time and a signal intensity of a frequency, to be superimposed on a corresponding brain disease region on a brain image, for each specific brain disease.

6. The brain function determination apparatus according to claim 2, wherein
the identification unit is configured to calculate, as the identification result, probabilities of each disease type of each brain disease and a healthy state, based on an output of the deep learning model, and
the display control unit is configured to display the probabilities.

7. The brain function determination apparatus according to claim 1, further comprising:
a second acquisition unit configured to acquire brain function data measured by the measurement apparatus and having a disease label added, the disease label indicating content of one of a brain disease and a healthy state;
a second conversion unit configured to convert the brain function data acquired by the second acquisition unit, to converted data including information on at least a time and a space as dimensions; and
a learning unit configured to construct a deep learning model through a learning process based on the deep learning, using the second converted data to which the disease label is added, as an input.

8. The brain function determination apparatus according to claim 7, further comprising a standardization unit configured to perform a predetermined standardization process on the second converted data, wherein
the learning unit is configured to construct the deep learning model, using the second converted data subjected to the standardization process, as an input.

9. The brain function determination apparatus according to claim 1, wherein the brain function data includes electro-encephalography data and magneto-encephalography data.

10. The brain function determination apparatus according to claim 1, wherein the first conversion unit is configured to convert the brain function data acquired by the first acquisition unit, to the first converted data including information on a frequency as a dimension.

11. The brain function determination apparatus according to claim 1, wherein the deep learning model is constructed by the deep learning with a time series analysis function.

12. A brain function determination method comprising:
acquiring brain function data including a temporal change, indicating a brain function state measured by a measurement apparatus;
converting the acquired brain function data to first converted data including information on at least a time and a space as dimensions; and
performing an identification process of determining a brain disease and identifying a brain disease region, using the first converted data as an input of a deep learning model constructed by predetermined deep learning.

13. A non-transitory computer-readable medium including programmed instructions that cause a computer to execute:
acquiring brain function data including a temporal change, indicating a brain function state measured by a measurement apparatus;
converting the acquired brain function data to first converted data including information on at least a time and a space as dimensions; and
performing an identification process of determining a brain disease and identifying a brain disease region, using the first converted data as an input of a deep learning model constructed by predetermined deep learning.
